(19) United States
(12) Patent Application Publication
(10) Pub. No.: US 2004/0033205 A1
(43) Pub. Date: Feb. 19, 2004

(54) Method of whitening teeth

(75) Inventors: Robert Francis Date, Surrey (GB); Samantha Jane Price, Middlesex (GB); Donald James White JR., Fairfield, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 10/641,251

(22) Filed: Aug. 14, 2003

Related U.S. Application Data
(60) Provisional application No. 60/403,725, filed on Aug. 15, 2002.

(51) Int. Cl. A61K 7/20

(52) U.S. Cl. 424/53

(57) ABSTRACT

The present invention provides a method of whitening a person's teeth comprising treating the teeth with a whitening dentifrice and subsequently, but within a ten minute period thereafter, applying a self-supporting film of liquid bleaching product to said teeth, for a bleaching period of at least five minutes, preferably overnight. At the end of the bleaching period the whitening dentifrice can be used to remove residual bleaching product and the whole process repeated. The whitening dentifrice comprises a surface stain remover, selected from water-soluble polyphosphates, phosphonate polymers and dental abrasives having an RDA value greater than 120, which enhances the whitening effect. The bleaching product comprises a tooth bleaching agent and a liquid carrier and has a viscosity of less than 1000 mPa.s at a shear rate of 10s⁻¹ and a temperature of 25° C.

Kits comprising the whitening dentifrice and the liquid bleaching product are also provided. The methods and kits herein provide for an improved tooth whitening effect for home usage.

### CROSS REFERENCE TO RELATED APPLICATIONS

This application claims the benefit of U.S. Provisional Application No. 60/403725, filed Aug. 15, 2002.

### FIELD OF THE INVENTION

The present invention relates to methods and kits for providing an improved tooth whitening effect through home usage.

### BACKGROUND OF THE INVENTION

Many people desire improved whiteness for their teeth. Often, a sufficient whitening effect is provided by brushing with a whitening toothpaste, which typically employs abrasives to remove surface or ‘extrinsic’ stains. There are many products of this type on the market. Intrinsic staining, where the enamel itself is stained, can also cause teeth discoloration. In such cases, a bleach treatment is typically required to provide the whitening necessary.

Professional bleach treatments, designed for administration by a dental professional in which a peroxide gel within a tray formed to the user's teeth shape have long been known. Bleach treatments have also been adapted for home use by providing generically formed trays. The trays, however, can be cumbersome to wear. More recently, thin substrate films coated with a tooth whitening gel have been described in WO 98/55044. Products of this general design are now available on the market as Crest Whitestrips®. Whilst all these treatments are very effective they all involve wearing a delivery device of some sort which may be uncomfortable over a prolonged period or present safety concerns if worn whilst asleep. They are also relatively expensive.

Paint- or brush-on bleaching films or varnishes are also known, for example from EP 511 782 which discloses hydroalcoholic compositions comprising hydroxypropyl cellulose and carbamide peroxide. Nonaqueous film-forming tooth whitening compositions comprising silicone resins, organic solvents and sodium percarbonate are also known from WO 97/25968 and from WO 01/01940. The latter discloses compositions comprising silicone resins, organic solvents and sodium percarbonate. These brush on films are intuitive for a user to apply and the films can be left in place for a prolonged period, such as overnight.

Bleaching treatments have also been provided within the context of a whitening toothpaste. GB 2,289,841, for example, discloses pastes containing sodium chlorite and bromelain and U.S. Pat. No. 5,814,304 discloses a toothpaste comprising a peroxide compound. There have been many disclosures as well of dual phase peroxide dentifrice compositions which either address the problem of incompatibility of peroxides with other dentifrice components or use a non-peroxide phase to container an activator for the peroxide. U.S. Pat. No. 6,106,812, EP 535,816 and WO 97/21419 are representative of these. Peroxide incompatible components disclosed in these documents include silica and alumina abrasives, sodium tripolyphosphate antitartar agent and a manganese gluconate peroxide activator. U.S. Pat. No. 6,290,935 discloses a two phase composition in which a peroxide phase is supplemented by an activating phase comprising an iron implanted silicate clay. In the case where activators are used, the aim is often to address the relatively low levels of peroxide that can be stably stored and used in such home usage compositions.

Usually these documents relating to dual phase compositions teach that the components are co-dispensed onto a brush and are mixed together on brushing the teeth. U.S. Pat. No. 6,174,516 describes a sequential treatment in which heightened tooth whitening is obtained by first applying an alkaline rinse to the teeth and then, without water rinsing, brushing with a urea peroxide dentifrice. U.S. Pat. No. 5,122,635 describes a three part sequential treatment of a conditioning mouth rinse of acetic acid to remove pellicle, application of a peroxide gel for one to four minutes followed by rinsing and a final brush with a polishing and pigmenting cream whose purpose is to both abrade the teeth and to mask residual discolorations.

Despite all the foregoing, there remains a need for inexpensive, easy to use tooth whitening systems which nevertheless provide a high level of effectiveness. It is an object of the present invention, therefore, to provide a tooth whitening regimen which is easy to follow and provides enhanced effectiveness versus the known paint or brush on products.

It is a further object of the present invention to provide a tooth whitening regimen, of enhanced efficacy, which encourages user compliance by fitting with the user's normal dental hygiene practise. It is yet a further object of the present invention to provide kits suitable for use with such a regimen.

These and other objectives will become readily apparent from the detailed description that follows.

### SUMMARY OF THE INVENTION

The present invention provides a method of whitening a person's teeth comprising treating the teeth with a whitening dentifrice and subsequently, but within a ten minute period thereafter, applying a self-supporting film of liquid bleaching product to said teeth, for a bleaching period of at least about five minutes, preferably overnight. At the end of the bleaching period the whitening dentifrice can be used to help remove residual bleaching product and the whole process repeated. The whitening dentifrice comprises a surface stain remover, selected from water-soluble polyphosphates, phosphonate polymers and dental abrasives having an RDA value greater than about 120, which enhances the whitening effect. The bleaching product comprises a tooth bleaching agent and a liquid carrier and has a viscosity of less than about 1000 mPa.s at a shear rate of 10s⁻¹ and a temperature of 25° C.

Kits comprising the whitening dentifrice and the liquid bleaching product are also provided. The methods and kits herein provide for an improved tooth whitening effect for home usage, and for a convenient extended regimen that suits the user's normal dental hygiene practise.

### DETAILED DESCRIPTION OF THE INVENTION

While the specification concludes with claims, which particularly point out and distinctly claim the invention, it is believed the present invention will be better understood from the following description.

Unless specified otherwise, all percentages and ratios herein are by weight and all measurements are made at 25° C. All viscosity measurements herein are made, unless otherwise specified, at 25° C. and at a shear rate of 10s⁻¹, using a steady state flow method. A suitable measurement instrument for this purpose is a TA instrument AR2000 which can be supplied by TA Instruments of New Castle, Del., USA.

The Whitening Dentifrice

The whitening dentifrice is used to treat the teeth before applying the liquid bleaching product. Treatment is generally by dispensing a small amount of the dentifrice onto a regular toothbrush and brushing all of the teeth surfaces, generally for a period of from about thirty seconds to about two minutes. The user, the person whose teeth are being whitened, can generally be expected to expectorate at the end of the treatment and, usually, to rinse with water.

The term “dentifrice”, as used herein, means paste, gel, or liquid formulations unless otherwise specified. The whitening dentifrice is of conventional form, such as a paste or gel and can be a single, dual or multi phase preparation. By ‘phase’ here is meant a separately packaged or filled part of the dentifrice such as a stripe within a tube. A single phase may comprise a liquid carrier with one or more insoluble particles, such as of a dental abrasive, homogeneously or evenly dispersed within it. The dentifrice is used to treat the teeth in a conventional manner, typically by application of a conventional amount onto a brush followed by brushing of the teeth with the dentifrice for a period of several seconds to about two minutes and then expectorating and/or rinsing with water.

An essential component of the whitening dentifrice herein is a surface stain remover selected from water-soluble polyphosphates, phosphonate polymers and dental abrasives having an RDA value greater than about 120. These components provide effective stain removal during the treatment process and, particularly with the long chain polyphosphates and phosphonate polymers herein, provide some surface residuality which, it is believed, enables them to provide an activating effect for the subsequently applied bleaching product. The selected surface stain removers further provide a slick, smooth feeling after brushing of the teeth, which enhances the overall usage experience, and prepare the tooth surface for an even coating with the bleaching product. Water-soluble polyphosphates and phosphonate polymers are preferred as surface stain removers and may be used in admixture.

A preferred class of surface stain removers is water-soluble polyphosphates. The water-soluble polyphosphates useful herein consist of three or more phosphate molecules arranged primarily in a linear configuration, although some cyclic derivatives may be present. Preferred polyphosphates herein are those having a chain length of at least 6, more preferably at least 10, typically present as their fully neutralised alkali metal salts. The polyphosphate salts include tetrapolyphosphate and hexametaphosphate, among others. Polyphosphates larger than tetrapolyphosphate usually occur as amorphous glassy materials. Preferred in this invention are the linear “glassy” polyphosphates having the formula XO(XPO3)ₙX where X is sodium or potassium and n averages from about 6 to about 125. Suitable polyphosphates are commercially available as Sodaphos (n˜6), Hexaphos (n˜13), and Glass H (n˜21) from Astaris, LLC, Creve Coeur IV, 622 Emerson Road, St. Louis, Mo. 63141. These polyphosphates may be used alone or in any combination thereof.

A further preferred class of surface stain removers is phosphonate polymers, particularly phosphonate copolymers. Examples of phosphonate copolymers are the diphosphonate-derivatized polymers in U.S. Pat. No. 5,011,913 to Benedict et al. A preferred polymer is diphosphonate modified polyacrylic acid. Suitable phosphonate-containing polymers such as shown below are described in U.S. Pat. No. 5,980,776 to Zakikhani, et al.

1. Co-polymer of acrylic acid and diphosphonic acid with structure:

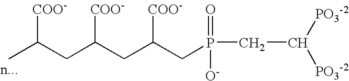

2. Co-polymer of acrylic acid and vinylphosphonic acid with structure:

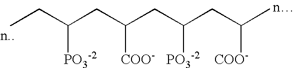

3. Co-polymer of methacrylic acid and vinylphosphonic acid with structure:

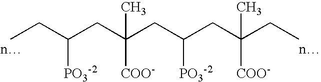

4. Co-polymer of acrylic acid and vinyldiphosphonic acid with structure:

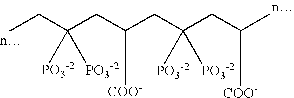

An alternate surface stain remover herein is a dental abrasive having an RDA (Radioactive Dentin Abrasion) value greater than 120, preferably from about 130 to about 250, more preferably from about 150 to about 200. The Radioactive Dentin Abrasion is a measure of the abrasiveness of the abrasive when incorporated into a dentifrice. The RDA values are determined according to the method set forth by Hefferren, Journal of Dental Research, July-August 1976, pp. 563-573, and described in Wason, U.S. Pat. Nos. 4,340,583, 4,420,312 and 4,421,527. The abrasive preferably has a mean value particle size in the range of about 5 to about 15 microns, preferably from about 8 to about 12 microns. The mean value particle size is measured using a Microtrac II Particle Analyzer apparatus, from Leeds and Northrup. Specifically, a laser beam is projected through a transparent cell which contains a stream of moving particles suspended in a liquid. Lights rays which strike the particles are scattered through angles which are inversely proportional to their sizes. The photodetector array measures the quantity of light at several predetermined angles. Electrical signals proportional to the measured light flux values are then processed by a microcomputer system to form a multi-channel histogram of the particle size distribution.

The chemical types of abrasives useful in the present invention can be those conventional in the art, such as silicas and aluminas. Preferred are precipitated silicas, especially those silicas with good fluoride compatibility, in particular compatibility with sodium fluoride and stannous fluoride sources.

The preferred precipitated silicas of the invention are low structure silicas in accordance with the definitions set forth in the J. Soc. Cosmet. Chem. 29, 497-521 (August, 1978), and Pigment Handbook: Volume 1, Properties and Economics, 2nd Edition, Edited by Peter A. Lewis, John Wiley & Sons, Inc., 1988, p. 139-159.

Mixtures of the polyphosphate and dental abrasive may also be used. Useful amounts of the surface stain remover range from as low as about 0.1% up to about 50% of the dentifrice. Preferred amounts are from about 5% up to about 35%, and more preferably from about 15% up to about 30%, of the dentifrice. Whilst the mechanism is not fully understood it is believed that the benefit is at least partly achieved through activation of the tooth bleaching agent of the liquid bleaching product by surface stain remover adhered or adsorbed to the tooth surface.

Optional dentifrice ingredients include materials such as surfactants, fluoride ion sources, additional anticalculus agents, buffers, other abrasive materials, thickening materials, humectants, water, flavours, sweetening agents, xylitol, colouring agents, and mixtures thereof.

One of the preferred optional agents of the dentifrice of the present invention is a surfactant. The surfactant can be present in the dentifrice of the present invention at from about 0.1% to about 2.5%, preferably from about 0.3% to about 2.5% and most preferably from about 0.5% to about 2.0% by weight of the dentifrice. Useful surfactant types include anionic, nonionic and betaine surfactants. Cationic surfactants can also be used though care needs to be taken over their compatibility with other typical dentifrice ingredients.

Anionic surfactants useful herein include the water-soluble salts of alkyl sulfates having from 10 to 18 carbon atoms in the alkyl radical and the water-soluble salts of sulfonated monoglycerides of fatty acids having from 10 to 18 carbon atoms. Sodium lauryl sulfate and sodium coconut monoglyceride sulfonates are examples of anionic surfactants of this type. Also useful herein are sarcosinate surfactants, isethionate surfactants and taurate surfactants, such as lauroyl sarcosinate, myristoyl sarcosinate, palmitoyl sarcosinate, stearoyl sarcosinate and oleoyl sarcosinate. All of the foregoing are generally used as their are alkali metal or ammonium salts.

Examples of suitable nonionic surfactants include the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials. Preferred betaine surfactants are disclosed in U.S. Pat. No. 5,180,577 to Polefka et al., issued Jan. 19, 1993. Typical alkyl dimethyl betaines include decyl betaine or 2-(N-decyl-N,N-dimethylammonio) acetate, coco betaine or 2-(N-coc-N, N-dimethyl ammonio) acetate, myristyl betaine, palmityl betaine, lauryl betaine, cetyl betaine, cetyl betaine, stearyl betaine, etc. The amidobetaines are exemplified by cocoamidoethyl betaine, cocoamidopropyl betaine, lauramidopropyl betaine and the like. The betaines of choice are preferably the cocoamidopropyl betaine and, more preferably, the lauramido propyl betaine.

Another preferred optional agent is a chelating agent. Chelating agents are able to complex calcium found in the cell walls of the bacteria. Chelating agents can also disrupt plaque by removing calcium from the calcium bridges, which help hold this biomass intact. Preferred chelating agents have a calcium binding constant of about 10¹ to 10⁵ to provide improved cleaning with reduced plaque and calculus formation.

Useful chelating agents herein include tartaric acid and pharmaceutically-acceptable salts thereof, citric acid and alkali metal citrates and mixtures thereof. Sodium and potassium citrate are the preferred alkali metal citrates, with sodium citrate being the most preferred. Also preferred is a citric acid/alkali metal citrate combination. Preferred herein are alkali metal salts of tartaric acid. Most preferred for use herein are disodium tartrate, dipotassium tartrate, sodium potassium tartrate, sodium hydrogen tartrate and potassium hydrogen tartrate. The amounts of chelating agent suitable for use in the present dentifrice are about 0.1% to about 2.5%, preferably from about 0.5% to about 2.5% and more preferably from about 1.0% to about 2.5%. The tartaric acid salt chelating agent can be used alone or in combination with other optional chelating agents.

Another group of agents suitable for use as chelating agents in the present invention are the soluble pyrophosphates. The pyrophosphate salts used in the present compositions can be any of the alkali metal pyrophosphate salts. Specific salts include tetra alkali metal pyrophosphate, dialkali metal diacid pyrophosphate, trialkali metal monoacid pyrophosphate and mixtures thereof, wherein the alkali metals are preferably sodium or potassium. The salts are useful in both their hydrated and unhydrated forms. An effective amount of pyrophosphate salt useful in the present composition is generally enough to provide at least 1.0% pyrophosphate ion, preferably from about 1.5% to about 6%, more preferably from about 3.5% to about 6% of such ions. It is to be appreciated that the level of pyrophosphate ions is that capable of being provided to the composition (i.e., the theoretical amount at an appropriate pH) and that pyrophosphate forms other than P₂O₇⁻⁴ (e.g., (HP₂O₇⁻³)) may be present when a final product pH is established.

Still another possible group of chelating agents suitable for use in the dentifrice are the anionic polymeric polycarboxylates. Such materials are well known in the art, being employed in the form of their free acids or partially or preferably fully neutralized water soluble alkali metal (e.g. potassium and preferably sodium) or ammonium salts. Preferred are 1:4 to 4:1 copolymers of maleic anhydride or acid with another polymerizable ethylenically unsaturated monomer, preferably methyl vinyl ether (methoxyethylene) having a molecular weight (M.W.) of about 30,000 to about 1,000,000. These copolymers are available for example as Gantrez AN 139 (M.W. 500,000), AN 119 (M.W. 250,000) and preferably S-97 Pharmaceutical Grade (M.W. 70,000), of GAF Chemicals Corporation.

It is preferred to have a water-soluble fluoride compound in the dentifrice in an amount sufficient to give a fluoride ion concentration in the composition of from about 0.0025% to about 0.5% by weight, to provide additional anticaries effectiveness. A wide variety of fluoride ion-yielding materials can be employed as sources of soluble fluoride in the present compositions. Representative fluoride ion sources include stannous fluoride, sodium fluoride, potassium fluoride, sodium monofluorophosphate and many others. Stannous fluoride and sodium fluoride are particularly preferred, as well as mixtures thereof. If, however, sodium fluoride is used in combination with the long chain polyphosphate stain removers then it is preferably kept in a separate phase.

The whitening dentifrice herein may further include pigments and colorants routinely used in the cosmetic arts. Most preferred are those selected from the group consisting of titanium dioxide, bismuth oxychloride, zinc oxide and mixtures thereof.

In preparing toothpaste or gels, it is often necessary to add some thickening material to provide a desirable consistency of the composition, to provide desirable active release characteristics upon use, to provide shelf stability, and to provide stability of the composition, etc. Preferred thickening agents are carboxyvinyl polymers, carrageenan, hydroxyethyl cellulose, laponite and water soluble salts of cellulose ethers such as sodium carboxymethylcellulose and sodium carboxymethyl hydroxyethyl cellulose. Natural gums such as gum karaya, xanthan gum, gum arabic, and gum tragacanth can also be used. A preferred class of thickening or gelling agents includes the class of crosslinked homopolymers of acrylic acid known as carbomers. Carbomers are cornmercially available from B. F. Goodrich as the Carbopol® series. Particularly preferred Carbopols include Carbopol 934, 940, 941, 956, and mixtures thereof.

Another optional but preferred component of the topical, oral carriers of the whitening dentifrice is a humectant. The humectant serves to keep the dentifrice from hardening upon exposure to air, to give a moist feel to the mouth, and, for particular humectants, to impart a desirable sweetness of flavour. The humectant, on a pure humectant basis, generally comprises from about 5% to about 70%, preferably from about 15% to about 45%, by weight of the dentifrice herein. Suitable humectants include edible polyhydric alcohols such as glycerin, sorbitol, xylitol, butylene glycol, polyethylene glycol, and propylene glycol, especially sorbitol and glycerin.

Flavouring and sweetening agents are preferably also added to the dentifrice. Suitable flavouring agents and sweetening agents are well known in the art.

Water employed in the preparation of the dentifrice should preferably be of low ion content and free of organic impurities. Water generally comprises from about 10% to about 50%, and preferably from about 20% to about 40%, by weight of the dentifrice herein. These amounts of water include the free water which is added plus that which is introduced with other materials, such as with sorbitol.

The pH of the present compositions can be adjusted, through the use of buffering agents to a preferred range of about 4.5 to about 9.5, more preferably from about 7 to about 8. Suitable buffering agents include monosodium phosphate, trisodium phosphate, sodium hydroxide, sodium carbonate, sodium acid pyrophosphate, citric acid, and sodium citrate. The pH of the dentifrice is measured from a 3:1 aqueous slurry of dentifrice.

Other optional agents that may be used in the present dentifrice include dimethicone copolyol plaque block agents selected from alkyl- and alkoxy-dimethicone copolyols, such as C12 to C20 alkyl dimethicone copolyols and mixtures thereof. Highly preferred is cetyl dimethicone copolyol marketed under the Trade Name Abil EM90. The dimethicone copolyols also aid in providing positive tooth feel benefits. These agents are described in more detail in WO 96/19191.

Other active agents, such as antimicrobial agents may also be employed. Included among such agents are water insoluble non-cationic antimicrobial agents such as halogenated diphenyl ethers, particularly triclosan and essential oils such as thymol. Water soluble antimicrobials include quaternary ammonium salts such as cetyl pyridinium chloride. Other antimicrobials such as copper bisglycinate, zinc citrate, and zinc lactate may also be included. Enzymes are another type of active that may be used in the present compositions. Useful enzymes include those that belong to the category of proteases, lytic enzymes, plaque matrix inhibitors and oxidases. Proteases include papain, pepsin, trypsin, ficin, bromelain; cell wall lytic enzymes include lysozyme; plaque matrix inhibitors include dextranses, mutanases; and oxidases include glucose oxidase, lactate oxidase, galactose oxidase, uric acid oxidase, peroxidases including horse radish peroxidase, myeloperoxidase, lactoperoxidase, chloroperoxidase. The oxidases also have whitening/cleaning activity, in addition to anti-microbial properties. Such agents are disclosed in U.S. Pat. No. 2,946,725, Jul. 26, 1960, to Norris et al. and in U.S. Pat. No. 4,051,234, Sep. 27, 1977 to Gieske et al.

The Liquid Bleaching Product

The liquid bleaching product contains a tooth bleaching agent which needs to be in contact with the teeth for a prolonged period, at least about five minutes, typically about thirty minutes or more, preferably for at least about one hour and more preferably for a much longer period such as overnight. In order to provide this prolonged contact the product needs to be capable of being applied to forming a self-supporting film when applied to the teeth. By “self-supporting film” is meant one which will stay in contact with the teeth without running off, without the use of a tray or other device to keep it in place. For this purpose the product needs to be capable of being applied evenly from an applicator and therefore suitably has a viscosity of less than about 1000 mPa.s, preferably less than about 500 mPa.s and more preferably less than about 150 mPa.s. In order that the product stays on the teeth once applied it preferably has a viscosity of at least about 1 mPa.s, and more preferably at least about 5 mPa.s.

An essential component of the liquid bleaching product is a tooth bleaching agent, to remove or bleach intrinsic or extrinsic stains on or in the tooth surfaces. Tooth bleaching agents are preferably selected from the group consisting of the peroxides, metal chlorites, perborates, percarbonates, peroxyacids, persulfates, and combinations thereof. Suitable peroxide compounds include hydrogen peroxide, urea peroxide, calcium peroxide, carbamide peroxide, and mixtures thereof. Suitable metal chlorites include calcium chlorite, barium chlorite, magnesium chlorite, lithium chlorite, sodium chlorite, and potassium chlorite. Additional bleaching agents include hypochlorite and chlorine dioxide. The preferred chlorite is sodium chlorite. A preferred percarbonate is sodium percarbonate. Preferred persulfates are oxones. The most preferred bleaching agents are hydrogen peroxide, sodium percarbonate, carbamide peroxide and mixtures thereof. The level of bleaching agent used is partly dependent on the available oxygen or chlorine respectively that the molecule is capable of providing to bleach the stain but is generally in the range from about 0.1% to about 35%, preferably from about 1% to about 25% and most preferably from about 3% to about 12% by weight of the liquid bleaching product.

An optional but highly desired component of the bleaching product is a film-forming polymer, which provides structure to the product, particularly once in place on the teeth. The term “film-forming polymer” herein means a polymer capable of forming, by itself alone or in the presence of a plasticizing agent, a visibly continuous film, whether porous or not. The film-forming polymer can be dissolved, or uniformly dispersed in the form of particles, in the liquid carrier of the bleaching product.

Many known thickeners are suitable for this purpose, such as starches, cellulose derivatives, polyethylene glycols and acrylate polymers such as carbomers. EP 511 782 discloses a suitable hydroxypropyl cellulose based product.

The film-forming polymer can further include materials such as polyurethanes; polyesters; alkyd resins; epoxyester resins; various silicone materials such as polysiloxanes, silicone gums and resins; and their mixtures. Preferred for use herein are silicones which, being generally water insoluble, are less prone to being washed off the teeth and also provide a pleasant lubricity to the film. Particularly preferred for use herein are organosiloxane resins.

Organosiloxane resins are highly crosslinked polymeric siloxane systems. The crosslinking is introduced through the incorporation of tri-functional and tetrafunctional silanes with mono-functional or di-functional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units, and hence, a sufficient level of crosslinking, such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen: silicon atoms is at least about 1.2:1.0.

In addition to the organosiloxane resins disclosed above, the liquid bleaching products of the present invention may further comprise a fluid diorganopolysiloxane-based polymer to be combined with the organosiloxane resins.

Useful fluid diorganopolysiloxane polymers are those comprising repeating units, where said units correspond to the formula (R₂SiO)ₙ where R is a monovalent radical containing from 1 to 6 carbon atoms, preferably selected from the group consisting of methyl, ethyl, propyl, isopropyl, butyl, isobutyl, t-butyl, amyl, hexyl, vinyl, allyl, cyclohexyl, amino alkyl, phenyl, fluoroalkyl and mixtures thereof. Fluid diorganopolysiloxane polymers employed in the present invention may contain one or more of these radicals as substituents on the siloxane polymer backbone.

Further description of the organosiloxane resins and fluid diorganopolysiloxane polymers herein is contained in PCT application WO 01/01940 which more broadly discloses preferred liquid bleaching products for use herein.

Generally the range of resin used in the present invention is from about 5% to about 70%, preferably from about 15% to about 45% and most preferably from about 20% to about 40%.

The bleaching product will generally comprise a solvent to impart the desired liquidity to the product and to provide homogeneity of structure. The solvent generally comprises from about 10% to about 90%, preferably from about 15% to about 80%, and more preferably from about 20% to about 70% of the liquid bleaching product. The solvent can be water, alcohol or a hydroalcoholic mixture. For some polymers such as the silicones described above an organic solvent is more suitable.

Preferred organic solvents have a boiling point of less than about 150° C., preferably less than about 100° C., and a solubility parameter of less than about 22 (MPa)^{0.5}. Solubility parameters are well known in the art and are readily available from tables, those used herein are SI Hildebrand values from Barton, Handbook of Solubility Parameters, CRC Press, 1983. The organic solvent is preferably selected from the group consisting of hydrocarbon oils, volatile silicones, non-hydrocarbon solvents, and mixtures thereof.

Hydrocarbon oils useful in the present invention include those having boiling points in the range of 60-150° C., more preferably hydrocarbon oils having from about C₆ to about C₁₀ chain lengths, most preferably C₇ to C₁₀ paraffins and isoparaffins. Most preferred is heptane.

The general classes of non-hydrocarbon solvents useful herein include esters, ketones, alcools, fluorocarbons and fluorocarbon ethers having boiling points in the range of 60 to 150° C. Non-hydrocarbon solvents or mixtures thereof particularly useful include those that are capable of solubilizing the resin and/or the diorganopolysiloxane-based polymer. Such solvents include but are not limited to butanone, ethyl acetate, propyl acetate, amyl acetate, ethyl butyrate, methyl nonafluoroisobutyl ether, methyl nonafluorobutyl ether, and mixtures thereof.

Preferred organic solvents are those selected from the group consisting of ethyl acetate, 2-butanone and heptane, more preferably 2-butanone (methyl ethyl ketone). Additional solvents may be used as required.

The liquid treatment product may further comprises other active agents and adjuvants such as structuring agents, chelants, stabilisers, fillers, flavours and the like. A broad range of active agents may be used, subject to compatibility with the polymers and resins herein, including oral and skin care benefit agents. Suitable active agents include teeth colour modifying substances such as pigments; anti-tartar agents, such as polyphosphates; fluoride ion sources such as sodium fluoride; anti-microbial agents such as triclosan; anti-inflammatory agents such as flurbiprofen or naproxen; nutrients such as zinc and vitamins; antioxidants such as ascorbic acid; H2 receptor antagonist compounds such as cimetidine and ranitidine; desensitising agents such as potassium nitrate; and antiviral actives such as inorganic stannous halides. A more complete listing of such actives is to be found in PCT publication WO 01/01940.

The liquid bleaching product may be packaged in any suitably protective package, such as a bottle providing sufficient product for the entire treatment period, but is conveniently provided in a unit dose, disposable laminate package. For the organic solvents herein a suitable laminate comprises an aluminium barrier layer and a polymer contact layer selected from polypropylene, polyimide and nylon 12. The unit dose package can comprise any suitable form such as sachets, peelable blisters and tear-open blisters. A preferred packaging form for the disposable package is a three seal sachet having a longitudinal seal and two transverse seals. The three seal sachet is preferably provided with a tear notch extending into one of the transverse seals. PCT application WO 95/01921 describes a suitable three seal sachet. The package is sized to hold the desired amount of liquid bleaching product forming the unit dose, taking into account any headspace desired. Suitable volumes of liquid bleaching product are from about 0.1 ml to about 10 mls, preferably from about 0.2 ml to about 2 mls, more preferably from about 0.2 ml to about 1 ml.

Methods of Use

In a first aspect the invention further to a method of whitening a person's teeth comprising treating the teeth with the whitening dentifrice described herein and, subsequently, but within about a fifteen minute, preferably about five minute period thereafter, applying a self-supporting film of the liquid bleaching product to the teeth and leaving the bleaching product in contact with the teeth for a bleaching period of at least five minutes. The mouth can be rinsed after the dentifrice treatment. The bleaching period is preferably at least about thirty minutes, more preferably at least one hour or more and more preferably several hours, such as overnight. It has been found that the bleaching product application is easier if the teeth are dried after treatment with the whitening dentifrice and before applying the bleaching product. The bleaching product is conveniently applied to the teeth by brushing with a soft brush. To avoid the applied film of liquid bleaching product being rubbed off onto the oral soft tissues such as lips, tongue and buccal cheek surfaces, especially the lips and tongue, it is generally necessary to keep those tissues away from the teeth until the film dries. A period of from about thirty seconds to about a minute is generally sufficient. A lip retractor, as described in PCT publication WO/02/07636, to hold back the lips whilst the liquid bleaching product is being applied, can be helpful in this respect. Dental rolls placed between the lips and the gum are also helpful and have the added advantage of keeping saliva away from the treated teeth surfaces whilst the bleaching product film dries. At the end of the bleaching period the teeth can be brushed with the dentifrice again to remove residual film and loosened stain.

The method above is intended for an extended regimen and is preferably repeated on a daily basis for a period of from about three to about twenty-eight days, preferably from seven to twenty-one days, more preferably for fourteen days. A convenient regimen is for the user to clean his or her teeth with the whitening dentifrice at night, in a usual manner before going to bed and then, having rinsed and dried the teeth, to apply the liquid bleaching product. After allowing a short period for the bleaching product to dry, the user can then go to sleep and brush again with the whitening dentifrice in the morning. If desired, a further application of the bleaching product can also be made at this time. Noticeable whiteness improvements can be seen within a few days, though two weeks or more may be more suitable for many people. Usage instructions on how to follow the required method will generally be included in a kit providing appropriate materials.

Kits

The present invention also provides kits comprising a packaged dentifrice as herein described, and a separately packaged, liquid bleaching product as herein described. The kit preferably comprises at least one applicator for the liquid bleaching product and preferably a plurality of applicators since after use of an applicator it may be clogged by dried bleaching product and be unsuitable for re-use. An applicator may comprise a brush having an elongated handle or a replaceable brush portion to be used with a reusable handle. A sponge or swab can also substitute the brush. In a preferred embodiment the kit comprises the bleaching product in unit dose form, wherein ‘unit dose’ means just sufficient product for one application of the method. A kit preferably comprises one applicator per unit dose product and the number of applicators and unit dose products provided in a kit can vary from about 3 to about 28. Most preferably a kit contains fourteen applicators and fourteen unit dose products to suit a two week, daily use regimen. The kits herein may further comprise additional elements selected from the group consisting of a lip retractor, a toothbrush and a plurality of dental rolls and will generally be packaged in a printed outer carton enclosing all the kit elements. The outer carton may be printed with the method details to help customers to assess its suitability before purchase.

### EXAMPLES

The following examples further describe and demonstrate embodiments within the scope of the present invention. These examples are given solely for the purpose of illustration and are not to be construed as limitations of the present invention as many variations thereof are possible without departing from the spirit and scope. All percentages used herein are by weight of the composition unless otherwise indicated.

The table below lists the formula for a representative whitening dentifrice product for use in the present invention. The dentifrice comprises two phases which are packaged in equal amounts in separate compartments of a suitable container and co-dispensed in two streams. The dual phase nature of the dentifrice allows, in particular, the sodium polyphosphate to be stably stored without risk of fluoride mediated hydrolysis.

| | Phase A | Phase B |
|---|---|---|
| Ingredient | % w/w | % w/w |
| Glycerin | 42.7 | 30.0 |
| Sodium fluoride | 0.00 | 0.64 |
| Sodium polyphosphate | 14.0 | 0.0 |
| Silica, precipitated | 24.0 | 20.0 |
| Sodium alkyl sulfate | 1.12 | 0.56 |
| Xanthan gum | 0.20 | 0.10 |
| Polyethylene glycol 300 | 6.66 | — |
| Polyethylene glycol 600 | 2.00 | — |
| Titanium dioxide | 0.50 | — |
| Flavour | 1.10 | 1.10 |
| Sodium saccharin | 0.40 | 0.40 |
| Sodium carboxymethyl cellulose | 0.10 | — |
| Poloxamer 407 | 0.50 | — |
| PEG 20 M | 0.50 | — |
| Sorbitol | — | 21.5 |
| Cocamidopropyl betaine | — | 0.87 |
| Carbomer | — | 1.00 |
| Sodium hydroxide | — | 0.80 |
| FD&C Blue No. 1 | — | 0.003 |
| Water | to 100% | to 100% |

The formula for an alternate, single phase whitening dentifrice, comprising a silica stain remover, is set out below:

| Material | % |
|---|---|
| Sorbitol solution (70%) usp | 24.96 |
| Silica-Zeodent 109¹ | 15.0 |
| Silica-Zeodent 118² | 15.0 |
| Thickening silica | 1.0 |
| Glycerin | 7.75 |
| Polyethylene glycol 300, nf (peg-6) | 6.0 |
| Tetrasodium pyrophosphate, anhydrous | 5.0 |
| Sodium alkyl solution (27.9%) | 5.0 |
| Sodium bicarbonate | 1.5 |
| Poloxamer 407, NF | 1.25 |
| Flavor | 1.1 |
| Titanium dioxide/carnauba wax prills | 1.0 |
| Sodium carboxymethyl cellulose | 0.75 |
| Sodium saccharin | 0.35 |
| Sodium carbonate, anhydrous | 0.50 |
| Sodium fluoride | 0.24 |
| Color | 0.05 |
| Water, purified usp | to 100% |

¹Precipitated silica from Huber having an RDA in excess of 130
²Precipitated silica from Huber having an RDA of 87

The table below lists the formula for a representative liquid bleaching product for use in the present invention. Other representative liquid treatment products are disclosed in PCT publication WO 01/01940.

| Material | % |
|---|---|
| Blue pigment, 15% dispersion in polydimethylsiloxane | 0.05 |
| SE30³ premix-9.76% SE30³ in DC200/20¹ | 10.25 |
| DC200/12500¹ | 1.0 |
| DC200/20¹ | 9.2 |
| 2-butanone | 3.0 |
| Ethyl acetate | 5.0 |
| MQ resin² | 32.5 |
| Flavour | 2.0 |
| Sodium bicarbonate | 8.0 |
| Aluminum hydroxide | 6.5 |
| Fumed silica | 3.5 |
| Sodium Percarbonate | 19.0 |

¹Polydimethylsiloxanes from Dow Corning
²Organosiloxane resin from General Electric
³A dimethicone gum from General Electric

0.5 ml of the liquid bleaching product is packaged in a sealed sachet to form a unit dose liquid bleaching product. Fourteen such unit dose sachets and fourteen brush applicators are enclosed in a display carton, along with a tube of either of the whitening dentifrice listed in the tables above, thereby forming a kit. The carton further includes the usage instructions:

| | |
|---|---|
| 1. | Before retiring to sleep, brush your teeth with the enclosed whitening |
| | dentifrice using your usual toothbrush |
| 2. | Dry your teeth |
| 3. | Open a sachet of the liquid bleaching product |
| 4. | Pick up a drop of the liquid bleaching product on one of the enclosed |
| | brush applicators and brush onto your teeth |
| 5. | Keep your mouth open for 60 seconds to allow the whitening film to |
| | form |
| 6. | Leave overnight and brush off with the whitening dentifrice as normal |
| | in the morning |
| 7. | Repeat daily for fourteen days |

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of whitening a person's teeth comprising:
a) treating the teeth with a whitening dentifrice comprising a surface stain remover selected from water-soluble polyphosphates, phosphonate polymers and dental abrasives having an RDA value greater than about 120; and
b) subsequently, but within a fifteen minute period thereafter, applying a self-supporting film of liquid bleaching product to said teeth and leaving the bleaching product in contact with the teeth for a bleaching period of at least about five minutes, the bleaching product comprising:
i) a tooth bleaching agent; and
ii) a liquid carrier, and having a viscosity of less than about 1000 mPa.s at a shear rate of 10⁻¹ and a temperature of 25° C.

2. A method according to claim 1 wherein the tooth bleaching agent is selected from peroxides, metal chlorites, perborates, percarbonates, peroxyacids, persulfates, and combinations thereof.

3. A method according to claim 2 wherein the tooth bleaching agent is selected from hydrogen peroxide, sodium percarbonate, carbamide peroxide and mixtures thereof.

4. A method according to claim 1 wherein the liquid carrier comprises a film-forming polymer selected from silicones, acrylate polymers, polyethylene glycols and cellulose derivatives.

5. A method according to claim 4 wherein the film-forming polymer comprises a cross-linked silicone resin.

6. A method according to claim 1 wherein the surface stain remover comprises a water-soluble linear polyphosphate having a chain length of at least 3.

7. A method according to claim 1 in which the film of liquid bleaching product is applied to the teeth by brushing.

8. A method according to claim 1 in which oral soft tissues are kept away from the teeth for a period of from about thirty seconds to about a minute to enable the applied film of liquid bleaching product to dry.

9. A method according to claim 8 in which the oral soft tissues are kept away from the teeth using a lip retractor or dental rolls.

10. A method according to claim 1 which further comprises the step of brushing the teeth with the whitening dentifrice at the end of the bleaching period.

11. A method according to claim 1 wherein the bleaching period is at least about thirty minutes.

12. A method according to claim 1 wherein steps a) and b) are repeated daily for a period of from about three to about twenty-eight days.

13. A method according to claim 1 wherein the teeth are dried after brushing with the whitening dentifrice and before applying the bleaching product.

14. A tooth whitening kit comprising:
a) a packaged dentifrice comprising a surface stain remover selected from water-soluble polyphosphates and dental abrasives having an RDA value greater than about 120;
b) a separately packaged, liquid bleaching product comprising:
i) a tooth bleaching agent; and
ii) a liquid carrier, and having a viscosity of less than about 1000 mPa.s at a shear rate of 10s⁻¹ and a temperature of 25° C.

15. A kit according to claim 14 which further comprises an applicator for the liquid bleaching product.

16. A kit according to claim 14 which further comprises additional elements selected from the group consisting of a lip retractor, a toothbrush and a plurality of dental rolls.

17. A kit according to claim 14 which comprises from 3 to 28 applicators and from 3 to 28 unit dose packages of the liquid bleaching product.

18. A kit according to claim 14 which further comprises instructions on how to comply with a tooth whitening method according to claim 1.

19. A method according to claim 1 wherein the surface stain remover comprises a water-soluble linear polyphosphate having a chain length of at least 6.

20. A method according to claim 1 wherein the surface stain remover comprises a water-soluble linear polyphosphate having a chain length of at least 10.

21. A method according to claim 1 wherein the bleaching period is one hour or more.

22. A method according to claim 1 wherein the bleaching period is overnight.

23. A method according to claim 1 wherein steps a) and b) are repeated daily for a period of from about seven to about twenty-one days.

24. A method according to claim 1 wherein steps a) and b) are repeated daily for a period of about fourteen days.
